(19) United States
(12) Patent Application Publication
(10) Pub. No.: US 2009/0111139 A1
(43) Pub. Date: Apr. 30, 2009

(54) Diagnostic technique for determining oncogenic signature indicative of tumorous growth

(75) Inventors: Kenneth Bloom, Laguna Niguel, CA (US); Gary Fogel, San Diego, CA (US); Jose DeLa Torre Bueno, Vista, CA (US)

Correspondence Address: Law Office of Scott C Harris Inc, PO Box 1389, Rancho Santa Fe, CA, 92067, US

(73) Assignee: CLARIENT, INC., Aliso Viejo, CA (US)

(21) Appl. No.: 11/929,858

(22) Filed: Oct. 30, 2007

(51) Int. Cl. C12Q 1/02; G06G 7/58

(52) U.S. Cl. 435/29; 703/11

(57) ABSTRACT

A priori knowledge is obtained from known tumor samples, indicative of cellular pathways associated with those known tumor samples. Multiple pathways are found for each tumor. This becomes a priori knowledge. Later unknown tumor samples are then analyzed against the a priori knowledge to find the pathways etc within the unknown tumor samples. Multiple pathways are collected to form an oncogenic signature. The oncogenic signature is used to find a cocktail of multiple treatments that treats each of the multiple pathways.

### BACKGROUND

Cancer is often treated by determining information about a tumor, e.g. a cancer, and using that information as a diagnostic tool in an attempt to determine how to treat the cancer. Current diagnostic tools typically analyze where and how the tumor arose. For example, the “where” might be a determination of whether the tumor arose from a specified kind of tissue. This determination is made based on the rationale that some cancers from some kinds of tissues are believed more aggressive than others. Therefore, it has been believed that the determination of how the tumor arose may be useful in determining how to treat the tumor. For example, a high-grade tumor may be more difficult to diagnose, because of the difficulty in determining from where it arose.

Current diagnostic techniques hence often attempt to deduce the original site of the tumor.

### SUMMARY

The present application describes new techniques for determining information about a tumor.

An embodiment determines pathways responsible for cellular anomalies. Tumors are then characterized to determine multiple pathways that are associated with characteristics of that tumor, where the characteristics can be genes or over/under expression of the genes.

### BRIEF DESCRIPTION OF THE DRAWINGS

FIG. 1 shows analysis of cells with known characteristics to deduce pathways;

FIG. 2 illustrates a flowchart of operation of this analysis;

FIG. 3 shows analysis of a tumor cell to find which of the a priori pathways are present in the specific tumor cell; and

FIG. 4 shows a flowchart of operation of determining the treatment for the specific tumor cell.

### DETAILED DESCRIPTION

The present invention investigates cellular pathways that are activated in a tumor cell and forms a signature indicative of multiple such pathways.

The kinds of pathways being discussed herein are a group of pathways acting in concert, one after another, to activate cell division or inhibit cell division. For purposes of this application, the term pathway is the action from a surface receptor on the cell to some agent, such as molecule, protein, or the like. The path may change the shape of the protein, for example, and then modify some other protein, or form an enzyme. This in turn changes the behavior of something else in the cell. Cell signaling can be used to characterize actions that cause things to happen in the cell. For example, the signaling can represent a determination of what is causing the cell to divide when it should not be dividing.

The inventors believe that there are a small number of pathways, for example between 5 and 10 different pathways, that are responsible for most of the cellular anomalies that eventually become tumors.

This recognitions is based, at least in part, on noticing that some cancer drugs rarely completely cure cancer. For example, the Herceptin drug has a known mechanism, and a known pathway that it inhibits. Herceptin often slows down cancer, increasing the time of survival by some amount. However, it rarely actually cures the cancer. The inventors believe the only time that Herceptin actually cures a cancer is in the unusual case where the tumor was caused by only a single specific pathway activation.

The inventors recognize that two tumors that have the same pathways activated are more likely to respond to the same treatment even if those tumors have different origins.

According to an embodiment, a tumor cell is characterized to determine a group of different pathways that are activated in specific cell. A combination of all the different pathways forms a signature, here called an “oncogenic signature”. The signature represents the set of multiple different pathways that are activated in the specific tumor being investigated.

There is a known relationship between certain drugs and the pathways they inhibit. A specific y drug inhibits x pathway. The oncogenic signature represents a group of pathways. That signature can be converted to providing a group of drugs, one or more drugs for each pathway, the group of drugs collectively inhibiting each of the individual pathways. As an example, Herceptin is known to attack Her2. This single chemical, however, inhibits only a single pathway.

The present application describes finding multiple pathways that form an oncogenic signature, and thereby also finding finds a combination of drugs that can be used to treat the patient.

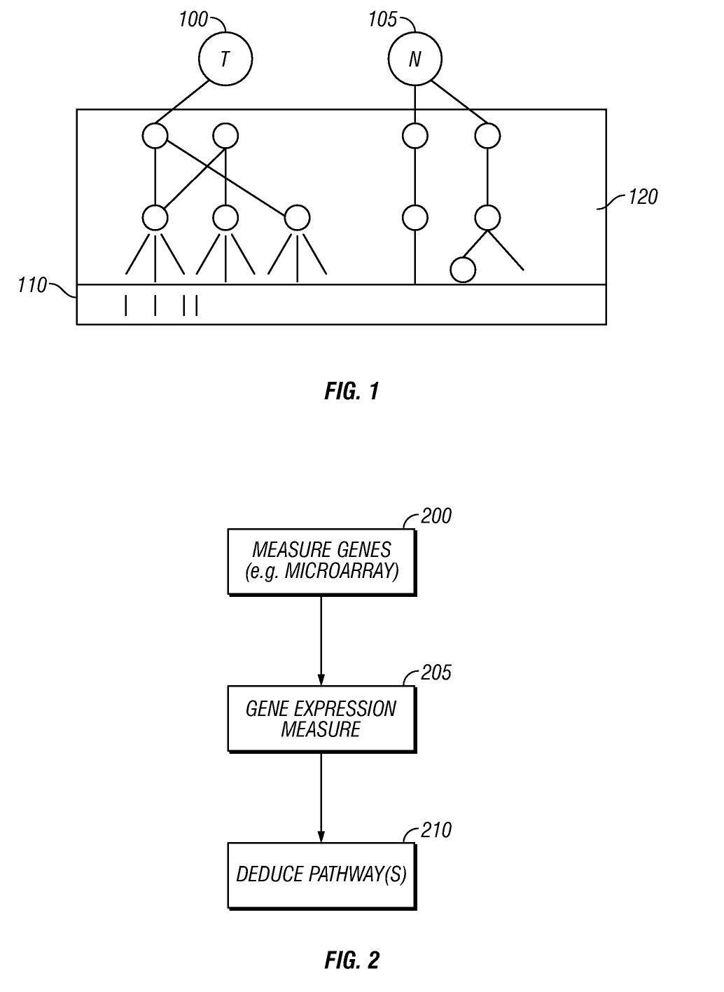

An initial determination of signatures may be carried out according to the illustration of FIG. 1 and according to the flowchart of FIG. 2. Known samples are analyzed, including a known tumor sample 100, and a known non-tumor sample 105. This characterization can use, for example, a gene microarray or other analysis vehicle at 200 to find results 110. The expression of the gene is determined at 205, e.g., overexpression of the gene, or underexpression of the gene. The samples are measured to determined “measures”, e.g., genes, proteins and the like.

The pathway(s) 120 can be deduced from those results, for example by using known information. For example, the literature includes many different studies that associate genes with the pathways that create those genes. Based on the results 110, the “hidden layers” 120 are postulated. The pathways will tend to cluster, based on this data.

There is likely to be a mixture of pathways between the upper layer 100 and the lower layer 110 forming the hidden layers between the known sample, and the measured products (genes, proteins, etc) It is also known in the literature to associate certain genes with certain pathways. For example, “oncogenic pathway signatures in human cancers as a guide to targeted therapies” nature 439 page 353, Jan. 19, 2006 illustrate known techniques of sorting genes according to their pathways. The system in FIG. 1 in essence forms a training set that allows finding pathways that are associated with these tumors. The observable genes are used with pathway knowledge to cluster those genes into pathways. The clustering may be inferred using statistical tools.

The multiple different pathways which are found for tumor cells form an a priori set of pathways that are used to later characterize a sample.

The number of tests on the known tumor samples may be at least ten times greater than a number of tests on the unknown tumor samples.

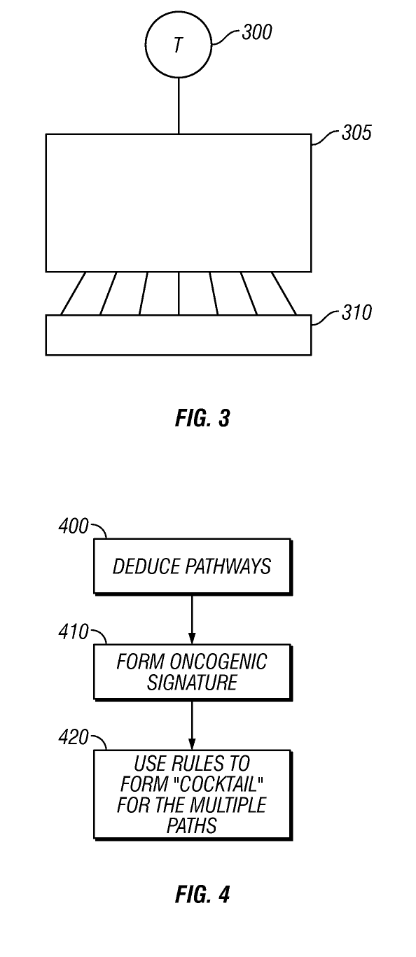

FIGS. 3 and 4 illustrate how the sample 300 is characterized. The sample 300 is analyzed, and used to find results 310. The results are analyzed at 400 using the a priori knowledge to determine multiple different pathways in the results. These multiple different signatures form an oncogenic signature at 410, indicative of multiple different pathways.

The pathways are each presumably pathways that were identified during the analysis at 210, that is, a priori paths. However, if there is a cluster that cannot be identified, then it may be deduced as being a new path, and analyzed according to the FIGS. 1 and 2 analysis.

Once the oncogenic signature is found, the therapies are found using a rule based lookup technique or other analogous technique. A rule based technique may define a set of rules, for example, of the form, if paths 1, 3 and 5 are on and paths 2 and 4 are off, then use drug cocktail ABC.

As explained above, if a known tumor shows no known pathways and/or no known drugs for inhibiting the pathways, this indicates that this must be a novel tumor which has no a priori data associated therewith. At this point, the patient's data is accessed using a microarray or other analysis device to find other genes and markers associated with the new path. In essence, a person whose tumor does not meet any of the known paths becomes a new clinical study.

Notice the significant difference between this technique and previous paradigms. The way things stand now, drugs are approved for a specific disease. With this technique, drugs would be approved for a specific pathway.

In an embodiment, the raw data from any known tumor or non-tumor sample will produce thousands of genes or gene products. As explained above, some genes will be indicative that pathway “A” has been followed. Other times combinations of genes, e.g., such as gene X in combination with gene Y will be indicative of pathway B. All of this can be based on studies or previously available literature. The raw data is used to form a data set of pathways, based on large amounts of data.

Further tests after the a priori knowledge is obtained then operates using a reduced subset of genes or gene products to find the active paths. For example, the reduced set may include hundreds of gene products, as compared with the initial determination which may analyze thousands of values. The paths are used to form the oncogenic signature for those paths that are active (410), and a prediction of a drug cocktail for the paths (420).

Models for mapping of input features such as genes and their expressions, protein, RNA, or other features to a decision of pathways may include such methods as artificial neural networks, fuzzy logic, support vector machines, hierarchical clustering, rule sets, finite state machines and hidden Markov models.

The techniques of optimization for models of features to pathways and/or signatures to drug selection can include population-based methods such as evolutionary computation, evolutionary algorithms, evolutionary programming, evolutionary strategies, genetic algorithms, genetic programming, enhanced colony optimization, particles swain optimization, differential evolution, associated evolutionary approaches that make use of variation and selection, as well as non-population-based approaches such as stimulated annealing and gradient descent based methods.

The rule sets may take any of a different number of different forms. For example, a simple set may be linearly separable, such as if there is input 1 equal to a value n; input 3 equal to a value y, then pathway x may be identified. The rule sets may be much more complex, such as if input 1*input 3/(square root of input 22)<3, then pathway x, else pathway why. The mapping may also use a linear mapping or a nonlinear mapping. For example, any other similar technique may alternatively be used, such as those disclosed in the above referenced article that show various ways in which gene expression patterns can be used to predict oncogenic pathways.

The general structure and techniques, and more specific embodiments which can be used to effect different ways of carrying out the more general goals are described herein.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, other techniques of determining the a priori knowledge may be used.

Also, the inventors intend that only those claims which use the words “means for” are intended to be interpreted under 35 USC 112, sixth paragraph. Moreover, no limitations from the specification are intended to be read into any claims, unless those limitations are expressly included in the claims.

The operations and/or flowcharts described herein may be carried out on a computer, or manually. If carried out on a computer, the computer may be any kind of computer, either general purpose, or some specific purpose computer such as a workstation. The computer may be an Intel (e.g., Pentium or Core 2 duo) or AMD based computer, running Windows XP or Linux, or may be a Macintosh computer. The computer may also be a handheld computer, such as a PDA, cellphone, or laptop. Moreover, the method steps and operations described herein can be carried out on a dedicated machine that does these functions.

The programs may be written in C or Python, or Java, Brew or any other programming language. The programs may be resident on a storage medium, e.g., magnetic or optical, e.g. the computer hard drive, a removable disk or media such as a memory stick or SD media, wired or wireless network based or Bluetooth based Network Attached Storage (NAS), or other removable medium or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

What is claimed is:

1. A method, comprising:
accessing information indicative of plural different cellular pathways, each of which plural different cellular pathways are responsible for at least one aspect of a tumor;
analyzing a specific tumor sample, to identify which of said plural different cellular pathways are active therein;
finding multiple of said different cellular pathways associated with said specific tumor sample, and forming an oncogenic signature indicative of said multiple different cellular pathways; and
using said oncogenic signature to determine treatments, where said treatments include multiple different treatments which collectively treat each of said multiple different cellular pathways.

2. A method as in claim 1, further comprising obtaining information about said cellular pathways from a gene array.

3. A method as in claim 1, wherein said analyzing comprises deducing said cellular pathways using known information.

4. A method as in claim 1, wherein said using comprises using a rule-based technique to look up a treatment based on which of said pathways are active, by determining at least multiple pathways which are active, and using said multiple pathways to access a rule in the form of if said multiple pathways are active, then use treatment A.

5. A method as in claim 1, further comprising identifying a specific tumor sample which has none of said cellular pathways being active, and further analyzing said specific tumor sample to determine new cellular pathways therein.

6. A method as in claim 1, wherein said obtaining comprises obtaining information associated with a first number of analyses, and said analyzing comprises obtaining information from a second number of analyses less than said first number now.

7. A method, comprising:
first analyzing plural known tumor samples to determine cellular pathways associated with said known tumor samples as a priori knowledge; and
second analyzing at least one unknown tumor sample, to determine plural different cellular pathways are responsible for at least one aspect of a tumor based on said a priori knowledge,
wherein a number of tests on said known tumor samples is at least ten times greater than a number of tests on said unknown tumor samples; and
using said plural different cellular pathways to find plural different treatments for said unknown tumor sample, each of which treatments is directed to a specific single one of said cellular pathways; and
providing said plural treatments to treat said unknown tumor sample.

8. A method as in claim 7, wherein said first and second analyzing comprises obtaining information about said cellular pathways from a gene array.

9. A method as in claim 7, wherein said using comprises using a rule-based technique to look up a treatment based on which of said pathways are active, by determining at least multiple pathways which are active, and using said multiple pathways to access a rule set in the form of if multiple pathways A and B are active, then use treatment C and D.

10. A method as in claim 7, further comprising identifying a specific tumor sample which has none of said cellular pathways being active, and further analyzing said specific tumor sample to determine new cellular pathways therein.

11. A method as in claim 1, wherein said obtaining comprises obtaining information associated with a first number of analyses, and said analyzing comprises obtaining information from a second number of analyses less than said first number now.

12. A testing apparatus, comprising:
a computer, having stored therein, information indicative of plural different cellular pathways, each of which plural different cellular pathways are responsible for at least one aspect of a tumor;
a gene analysis part, analyzing a specific tumor sample, to identify which of said plural different cellular pathways are active therein;
said computer finding multiple of said different cellular pathways associated with said specific tumor sample, and forming an oncogenic signature indicative of said multiple different cellular pathways, and using said oncogenic signature to determine treatments, where said treatments include multiple different treatments which collectively treat each of said multiple different cellular pathways.
